(19) United States
(12) Patent Application Publication
(10) Pub. No.: US 2006/0269475 A1
(43) Pub. Date: Nov. 30, 2006

(54) Multi-layer structure having a predetermined layer pattern including an agent

(76) Inventors: WonHyoung Ryu, Stanford, CA (US); Rainer J. Fasching, Mill Valley, CA (US); Friedrich B. Prinz, Woodside, CA (US); Ralph S. Greco, Stanford, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/402,651

(22) Filed: Apr. 11, 2006

Related U.S. Application Data
(60) Provisional application No. 60/670,483, filed on Apr. 11, 2005.

(51) Int. Cl. A61K 51/00; A61K 9/14

(52) U.S. Cl. 424/1.11; 424/486

(57) ABSTRACT

Improved controlled therapy is provided with a polymer multi-layer structure having a predetermined micro-fabricated spatial pattern (e.g., reservoirs and channels). More specifically, all geometrical details of the spatial pattern are substantially predetermined. The increased control of pattern geometry provided by the invention allows for improved control of therapy. In preferred embodiments, the polymer multi-layer structure of the invention is biodegradable, but has an in vivo lifetime that is greater than the duration of the therapy being provided. Thus, the geometrical pattern of the polymer structure that controls delivery of the therapy persists without significant change during therapy, and the structure degrades after completion of therapy. In this manner, possible interference of degradation by-products with therapy is minimized, and delivery of therapy does not depend on details of how degradation proceeds.

### CROSS REFERENCE TO RELATED APPLICATIONS

This application claims the benefit of U.S. provisional application 60/670,483, filed on Apr. 11, 2005, entitled “Multi Layered Thin Sheet Constructs for Non Implantable and Implantable Controlled Drug and/or Radiation Delivery Devices”, and hereby incorporated by reference in its entirety. This application also claims the benefit of U.S. application Ser. No. 11/078,907, filed on Mar. 11, 2005, and entitled “3-D Interconnected Multi-Layer Microstructure of Thermoplastic Materials”.

### FIELD OF THE INVENTION

This invention relates to controlled delivery of agent(s) for therapy and other applications.

### BACKGROUND

Controlled delivery of therapy has been of great interest in medicine for many years, especially in cases where it is undesirable or impractical to provide frequent doses of therapy. For example, timed-release tablets or capsules of various kinds have been developed to reduce dosage frequency, release ingested drugs in specific parts of the digestive system, and other variations. Representative examples include U.S. Pat. No. 6,207,197, U.S. Pat. No. 6,620,439, U.S. Pat. No. 5,672,359, U.S. Pat. No. 4,218,433, and U.S. Pat. No. 3,317,394. Such tablets tend to rely on bio-degradation of tablet materials to provide a more controlled release of drugs than would otherwise be obtained.

Another approach for providing controlled therapy is a device having multiple reservoirs of an agent to be delivered. For example, US 2004/0248320 considers such a device where each reservoir is individually electrically controllable such that a reservoir cap can be selectively disintegrated or permeabilized, thus releasing the agent. U.S. Pat. No. 6,010,492 and US 2006/0057737 also consider devices having reservoirs which can be independently actuated to control drug release. A passive device having a drug reservoir is considered in US 2005/0118229, where release is controlled by a composite nano-porous/micro-porous membrane covering the reservoir.

Controlled therapy by providing polymer multi-layers including a drug-loaded layer has also been considered, e.g., as in U.S. Pat. No. 6,322,815, U.S. Pat. No. 5,603,961, and U.S. Pat. No. 6,316,018. Such polymer multi-layers often include one or more porous layers. Porous layers can be loaded with one or more drugs in the pores and/or can be used to control the drug delivery rate. Representative examples include U.S. Pat. No. 5,605,696, U.S. Pat. No. 4,666,702, U.S. Pat. No. 5,656,296, U.S. Pat. No. 4,895,724, U.S. Pat. No. 4,525,340, U.S. Pat. No. 5,156,623, and U.S. Pat. No. 5,969,020.

Multi-layer drug-releasing constructs have found various applications, including vascular graft and stent covers (U.S. Pat. No. 6,702,849), drug delivery via a patch applied to mucosal tissue (US 2003/0219479), and transdermal drug delivery (U.S. Pat. No. 5,273,756 and U.S. Pat. No. 3,797,494).

Although it is clear that controlled drug delivery has been extensively investigated, not all issues have been completely resolved. For example, in cases where a drug is incorporated into a degradable structure to control delivery, it is necessary to ensure that the degradation products of the structure do not interfere with the drug being delivered. Furthermore, it can be difficult to control the drug release rate by controlling the degradation process. In cases where a porous polymer layer is used to hold drugs and/or to control the delivery rate, the delivery rate can depend sensitively on parameters of the porous layer (e.g., porosity, mean pore size, degradation rate) which are imperfectly controlled during fabrication. For example, two membranes made in different ways (or by different manufacturers) may have different drug delivery properties even if they nominally have the same pore size and porosity.

Accordingly, it would be an advance in the art to provide controlled therapy that is free from such undesirable complications.

### SUMMARY

The present invention provides improved controlled therapy with a polymer multi-layer structure having a micro-fabricated spatial pattern (e.g., reservoirs and channels). Preferably, the micro-fabricated spatial pattern on the polymer is a predetermined pattern. More specifically, the geometrical details of the spatial pattern are substantially predetermined, in sharp contrast to conventional porous polymer layers. In a conventional porous polymer layer, the pore size may be controlled by fabrication, but the detailed position of each pore is not predetermined. The increased control of pattern geometry provided by the invention allows for improved control of therapy. In preferred embodiments, the polymer multi-layer structure of the invention is biodegradable, but has an in vivo lifetime that is greater than the duration of the therapy being provided. It is preferred that the geometrical pattern of the polymer structure that controls delivery of the therapy persists without significant change during therapy, and the structure degrades after completion of therapy. In this manner, possible interference of degradation by-products with therapy is minimized, and delivery of therapy does not depend on details of how degradation proceeds.

Embodiments of the invention can provide many advantages. Solvent sensitive drugs can be employed, since exposure of drugs to solvents can be avoided. Since therapeutic agents are loaded into matrix layer voids, the loading capacity is independent of the solvability of the agent in the polymer. Loading of agents into the polymer matrix layer is not affected by miscibility, partitioning and/or aggregation behavior of the agent relative to the polymer. Thus high and uniform loading can more easily be achieved. Loading of agents can be performed after fabrication of the polymer multi-layer structure (e.g., shortly prior to use by an end user). Such loading is particularly useful for toxic, radioactive and/or unstable therapeutic agents. Loading can be customized, especially in cases where the agent(s) are in liquid form and loading is via capillary action. Multiple matrix layers can be employed in a modular manner to provide release of multiple agents. In such cases, fabrication is not affected by interactions between the agents, since they are loaded into separate layers. The generally planar shape of these polymer multi-layer structures is conducive to a wide variety of application and fabrication methods (e.g., wrapping, folding, rolling, bonding, lamination wrapping, and sewing). In particular, large sheets of agent-loaded polymer multi-layers can be fabricated to reduce cost. Device shape can be customized by an end user as needed.

Fully biodegradable micro-fabricated drug delivery systems can be fabricated. As indicated above, the encapsulation and matrix layers preferably degrade after therapy is complete, which eliminates any need for re-surgery in cases where an implant is employed. Release is controlled without relying on excipient properties, and can be customized at will by design (e.g., to provide zero order and/or pulsed release). The burst effect can be prevented by appropriate design of the encapsulation layer and/or barrier layer. Sequential delivery of multiple drugs can be provided. In a multi-layer device, the bottom matrix layers deliver drugs later than the top matrix layers. In a single layer device, regions of the matrix layer far from the encapsulation layer holes deliver drugs later than regions closer to the holes. Delivery mechanisms can be different for different drugs, even in the same device. For example, one agent can be diffusion limited, while delivery of another agent is osmosis driven. Sheet devices can directly provide therapy over a large area, as opposed to relying on transport within host tissue (e.g., micro-spheres or pellets). This is particularly relevant when the therapeutic agent is radioactive, since highly uniform radiation over a large area can be provided. The use of excipient polymers can be minimized, thereby minimizing inflammation or irritation due to degradation by-products. Degradation of polymers can be employed to enhance release in osmosis driven devices. In particular, retention of degradation by-products can be employed to increase osmotic pressure, thereby tending to maintain a constant drug delivery rate even as the drug concentration within the device begins to decrease.

Combined therapy can be provided. For example, a single polymer structure can release a chemical radio-sensitizer and also provide radiation therapy from a radioactive agent in sealed voids (e.g., for Brachy therapy). Polymer structures of the invention can be mounted on one or more surfaces of an implant, to provide local drug delivery between implant surface and body tissue.

The invention is applicable for providing a wide variety of therapies, including but not limited to the following examples: delivery of antibiotics for periodontitis; delivery of medication for glaucoma treatment; delivery of agents for skin treatment; transdermal delivery of drugs or medications; delivery of growth factors, peptides, or DNA for wound healing, skin tissue repair, peripheral or central nervous system repair, skeletal or muscle tissue repair, vascular tissue regeneration, and/or controlled differentiation of stem cells; delivery of pain relief agents and/or antibiotics for post-operative treatment; temporary or permanent implantation; and local delivery of anti-cancer medication, radio-sensitizer and/or radiation for cancer treatment.

### BRIEF DESCRIPTION OF THE DRAWINGS

FIGS. 1a-e show some encapsulation layers suitable for use in embodiments of the invention.

FIGS. 2a-d show some barrier layers suitable for use in embodiments of the invention.

FIGS. 3a-h show some matrix layers suitable for use in embodiments of the invention.

FIGS. 4a-g show some embodiments of the invention.

FIGS. 5a-c show an example of how an embodiment of the invention can operate in practice.

FIG. 6 shows a top view of an embodiment of the invention.

FIGS. 7a-b show drug release as a function of time for an embodiment of the invention compared to a control.

FIGS. 8a-b show an embodiment of the invention where drug-containing reservoirs are connected to an outer surface of a delivery device via channels.

FIG. 8c shows drug delivery rates for embodiments according to FIGS. 8a-b having different channel lengths.

FIG. 9a shows example of different channel shapes.

FIG. 9b shows examples of different reservoir configurations.

FIGS. 10a-b show an embodiment of the invention where the therapeutic agent is radioactive and the encapsulation layer is a solid layer having no through holes.

FIG. 10c shows dose vs. distance for the embodiment of FIGS. 10a-b.

### DETAILED DESCRIPTION

According to a first aspect of the invention, controlled therapy is provided by a structure including at least two polymer layers: a matrix layer and an encapsulation layer. The matrix layer is patterned such that it has voids, within which one or more therapeutic agents are disposed. In preferred embodiments, the geometrical details of the matrix layer spatial pattern are substantially predetermined. In particular, there are pattern parameters (e.g., void size, void shape, etc.) which are predetermined. In order to provide such predetermined patterns, microfabrication techniques can be employed to form the predetermined pattern in the matrix polymer layer. Suitable techniques for such microfabrication are described in US 2005/0206048, hereby incorporated by reference in its entirety. Other suitable techniques for pattern fabrication include embossing, laser machining, photo cross linking methods such as stereolithography, and casting. As indicated above, the fully predetermined pattern of the present invention is in sharp contrast to conventional drug-loaded porous layers, which are not completely predetermined. For example, a porous layer may have a specified average pore size and a specified average pore density, but the details of pore distribution and shape are not predetermined. Predetermined geometrical patterns in the matrix layer (and optionally in the encapsulation layer as well) can be used to provide improved control of a therapy being delivered.

In one aspect of the invention, the delivery device comprises a matrix layer with a geometrical pattern, where the term “geometrical” means that the spatial arrangement of voids or channels in the matrix layer is non-random. The term “non-random” means that the position of pores, voids, channels or reservoirs, as well as the distribution or shape of such pores, voids, channels or reservoirs, has a certain (i.e., 100%) probability of occurrence. In a further aspect the “non-random” characteristic can be in the encapsulation layer alternatively or concomitantly to the matrix layer, and/or barrier layer. Therefore, the non-random feature of the device provides for improved control of delivery of one or more therapeutic capable agents, thus ultimately improving control of therapy.

The encapsulation layer is disposed to cover the matrix layer spatial pattern. In some embodiments of the invention, the encapsulation layer is in contact with the matrix layer. In other embodiments, a barrier layer is disposed between and in contact with the encapsulation layer and the matrix layer. Typical matrix and encapsulation layer thicknesses are between about 50 μm and about 150 μm. Typical barrier layer thicknesses are between about 50 μm and about 200 μm.

The matrix layer, encapsulation layer and barrier layer (if present) can be selected from categories such as bio-absorbable polymers, non-absorbable polymers, water soluble polymers, and water insoluble polymers.

Suitable bio-absorbable polymers include but are not limited to: aliphatic polyesters, poly(amino acids), copoly(ether-esters), polyalkylene oxalates, polyamides, poly(iminocarbonates), polyorthoesters, polyoxaesters, polyamidoesters, polyoxaesters containing amine groups, poly(anhydrides), polyphosphazenes, polyoxaamides and polyoxaesters containing amines and/or amido groups, and blends thereof. Polyanhydrides from diacids of the form HOOC—C₆H₄—O— (CH₂)ₘO—C₆H₄—COOH where m is an integer in the range of 2 to 8 and copolymers thereof with aliphatic alpha-omega diacids of up to 12 carbons are also suitable.

Aliphatic polyesters include but are not limited to homopolymers and copolymers of lactide (which includes lactic acid, d-, l- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone, hydroxybutyrate (repeating units), hydroxyvalerate (repeating units), 1,4-dioxepan-2-one (including its dimer 1,5,8,12-tetraoxacyclotetradecan 7,14-dione), 1,5-dioxepan-2-one, 6,6-dimethyl-1,4-dioxan-2-one 2,5-diketomorpholine, pivalolactone, alpha, alpha diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof.

Suitable non-absorbable polymers include but are not limited to: poly(dimethylsiloxane), silicone elastomers, polyurethane, poly(tetrafluoroethylene), polyethylene, polysulfone, poly(methyl methacrylate), poly(2-hydroxyethyl methacrylate), polyacrylonitrile, polyamides, polypropylene, poly(vinyl chloride), poly(ethylene-co-(vinyl acetate)), polystyrene, poly(vinyl pyrrolidine).

Suitable water soluble polymers include but are not limited to: saccharides such as cellulose, chitin, dextran, proteins such as collagen and albumin, acrylates and acrylamides such as poly(acryl acid), polyacrylamide, and poly(1-hydroxyethyl methacrylate), and poly(ethylene glycol).

Suitable water insoluble polymers (and other layer materials) include but are not limited to: yellow wax, petrolatum cholesterol, stearyl alcohol, white wax, white petrolatum, methylparaben, propylparaben, sodium lauryl sulfate, propylene glycol, glycerogelatins, geling agents such as carbomer 934, cellulose derivatives, natural gums, penetration enhancers such as dimethyl sulfoxide, ethanol propylen glycol, glycerin, urea, glycerogelatins, coloring agents, lactose, stearic acid, starch glycolate, sugar, gelatin, fixed vegetable oils and fats, glycerin, propylene glycol, alcohol, ethyl oleate, isopropyl myristate, dimethyl acetamide, and mixtures or aqueous or oil based dispersions of these.

One aspect of the invention is to provide a modular approach for therapy control, where each layer performs specific functions. The encapsulation layer controls the amount of water that will be taken up from the surroundings and also controls the release of the therapeutic agent(s) from the matrix layer. Such controlled release is typically provided by through holes in the encapsulation layer when the therapeutic agent is a chemical agent. By controlling the permeability and opening size in the encapsulation layer, the release mechanism can be diffusion limited release, osmotic pressure driven release, or any combination of these mechanisms. Preferably, the encapsulation layer spatial pattern is a predetermined micro-structured pattern, as described above for the matrix layer. The pattern fabrication techniques described above in connection with the matrix layer pattern are also suitable for fabricating the encapsulation layer pattern. In cases where the therapeutic agent is a radioactive agent, the encapsulation layer typically includes no through holes.

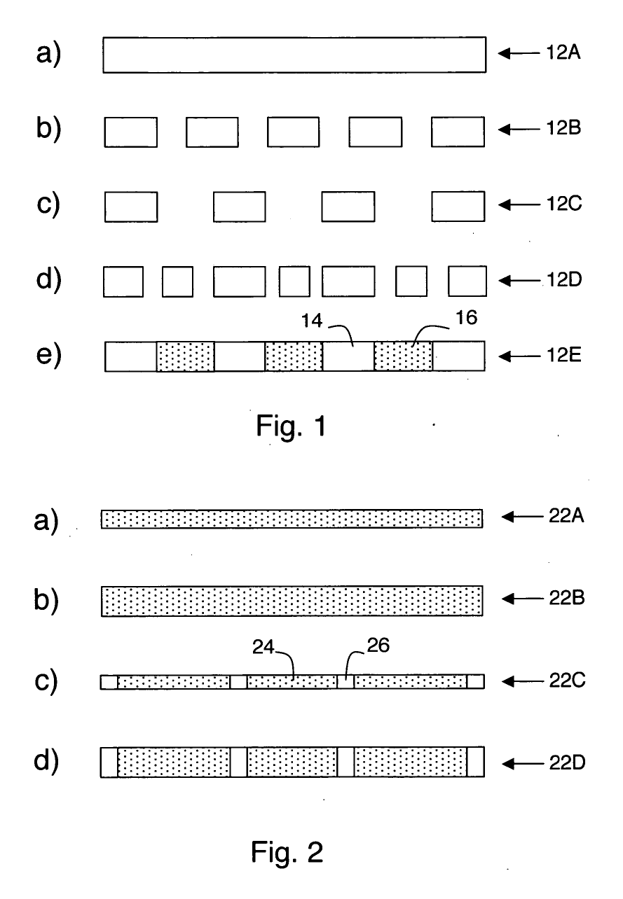

Examples of encapsulation layer patterns are shown on FIGS. 1a-e. Encapsulation layer 12A of FIG. 1a has no through holes, and is suitable in cases where the therapeutic agent is a radioactive agent that is not intended to be released while it is active. FIGS. 1b-d show encapsulation layers 12B, 12C and 12D having through holes with various sizes and densities. FIG. 1e shows an encapsulation layer 12E fabricated of a non-degrading or slowly degrading material 14, where through holes in material 14 are filled with a relatively rapidly degrading material 16.

The barrier layer (if present) can degrade partially or completely during therapy. For diffusion driven drug release, the drug can diffuse through the barrier layer to reach the encapsulation layer. Properties (e.g., degradation rate, diffusion rate) of the barrier layer can be selected to provide further control of drug delivery in addition to the control provided by the encapsulation layer. For osmotic pressure driven drug release, the barrier layer can degrade completely during therapy, such that a drug containing liquid is formed between the matrix layer and the encapsulation layer having high enough concentration to drive osmosis.

Examples of barrier layers are shown on FIGS. 2a-d. FIGS. 2a-b show barrier layers 22A and 22B having different thicknesses. FIG. 2c shows barrier layer 22C having pockets of a relatively rapidly degrading or solvable material 24 separated by a relatively slowly degrading material 26. Barrier layer 22D of FIG. 2d is similar to barrier layer 22C, except that the thickness is increased.

The matrix layer (or layers) acts as carriers for one or more therapeutic agents (e.g., drugs and/or radioactive material). Therapeutic agents are loaded into voids formed in the matrix layer as part of a predetermined pattern. Loading of agents into the matrix layer can be performed in various ways (e.g., micro dispensing, micro injection, powder compaction, screen-printing, ink jet printing, or sieving). For agents in liquid form, loading can rely on capillary action. In such cases, the microstructured matrix layer pattern is preferably in the form of a continuous micro-channel system as opposed to discrete reservoirs. Loading of agents into the structure can be performed before or after fabrication of the multi-layered structure.

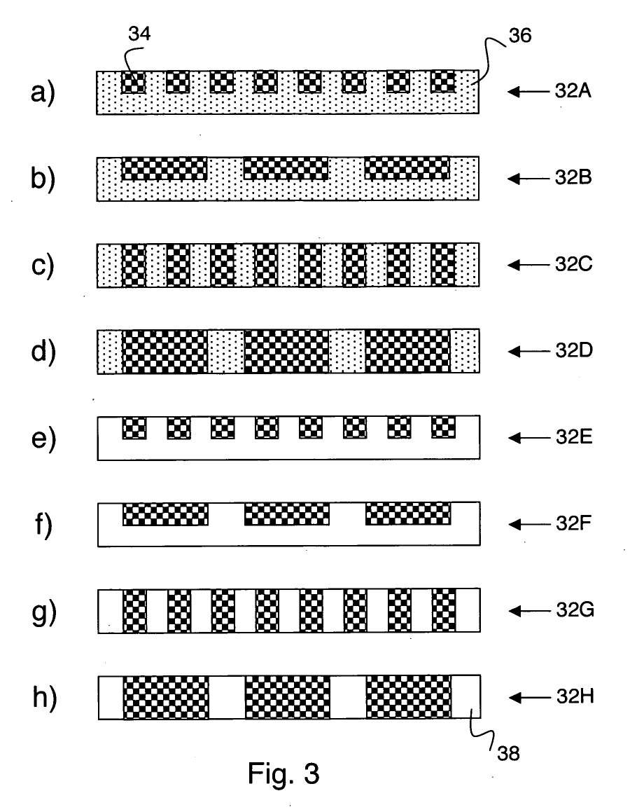

Examples of matrix layers are shown on FIG. 3a-h. Matrix layer 32A of FIG. 3a is fabricated of a relatively rapidly degrading or solvable material 36, where voids in material 36 are loaded with a therapeutic agent 34. Variations include changing void size and/or spacing (e.g., matrix layer 32B) and/or including through holes in the pattern (e.g., matrix layers 32C and 32D). More commonly, the matrix layer is fabricated of a relatively slowly degrading material 38, and matrix layers 32E, 32F, 32G and 32H correspond to layers 32A, 32B, 32C and 32D with this change of material.

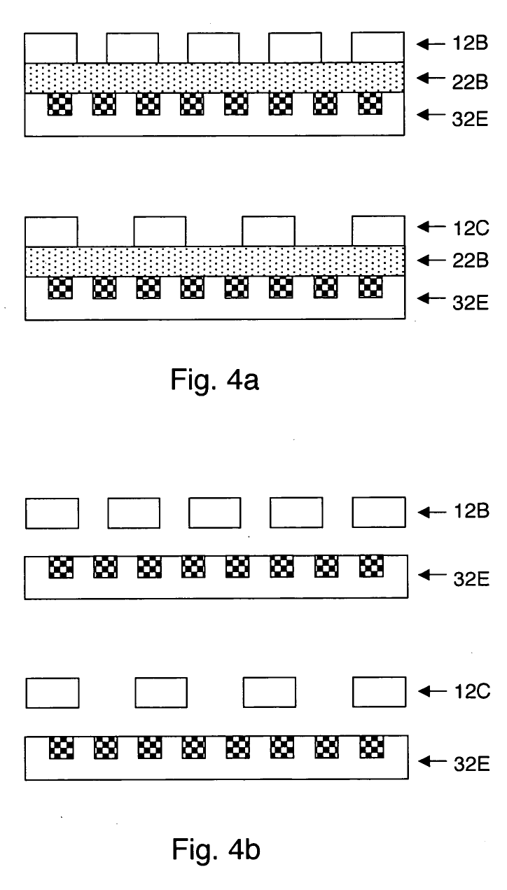

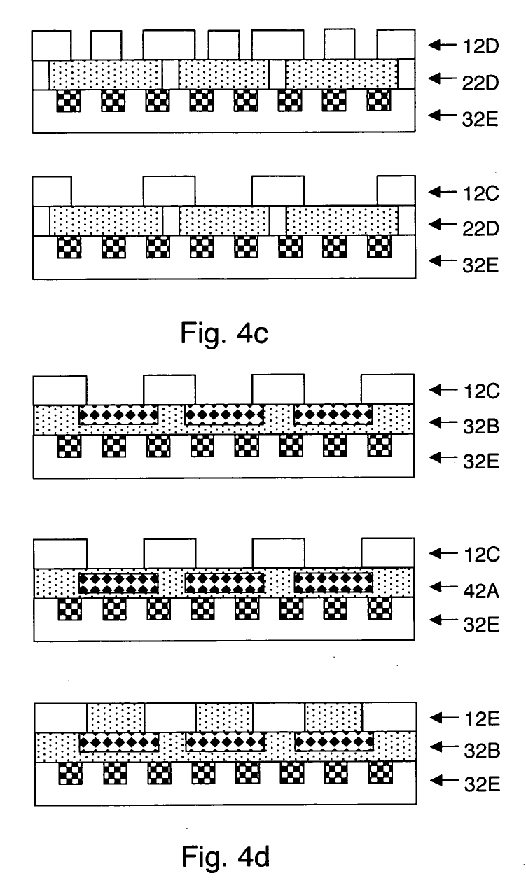

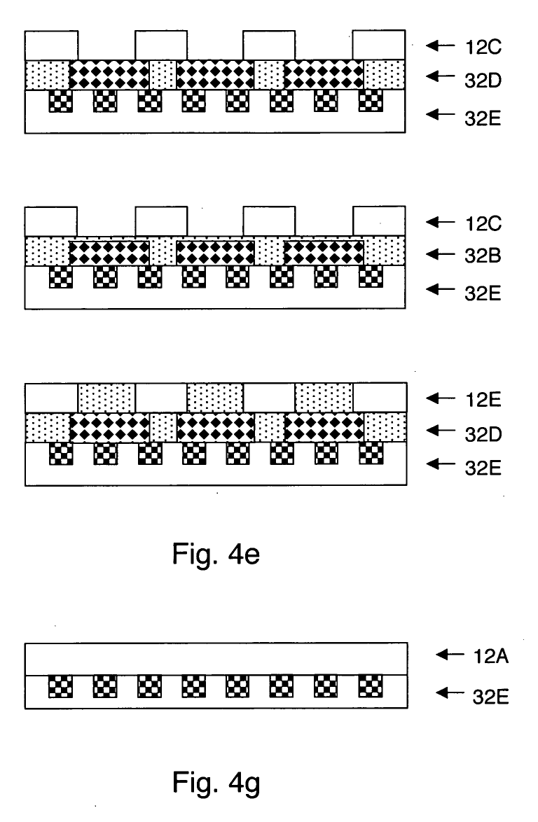

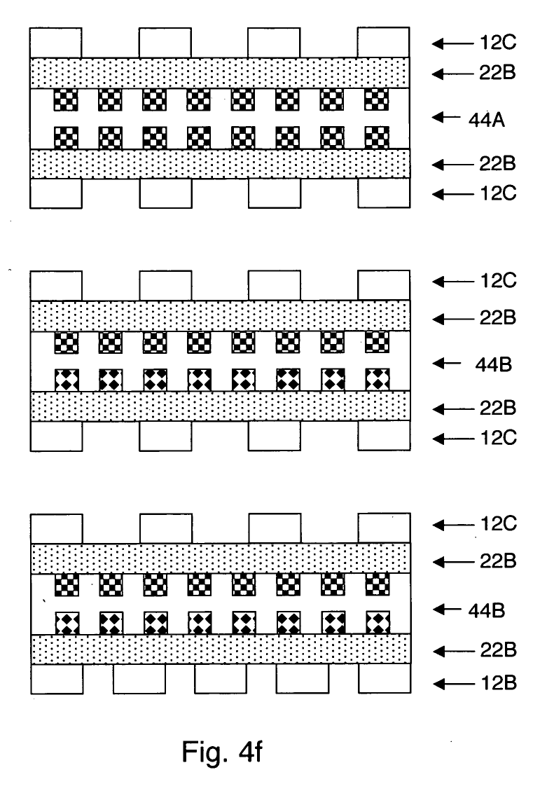

FIGS. 4a-g show some embodiments of the invention. The examples of FIG. 4a-g illustrate the modularity of embodiments of the invention. Individual variations of each layer can be employed to provide a wide variety of controlled therapy. Such structures can provide controlled release of both hydrophobic and hydrophilic drugs, and can provide controlled release of low molecular weight and high molecular weight drugs. Material and/or geometrical parameters of these structures can be selected to provide diffusion limited drug release, osmotic pressure driven drug release, or any combination of these mechanisms.

Constructs as in FIG. 4a having a matrix layer 32E, barrier layer 22B and encapsulation layer 12B or 12C are suitable for release of a drug at a constant rate. Release rate can be controlled by selecting the water permeability of the encapsulation layer, the size of the encapsulation layer through holes (e.g., large in layer 12C and small in layer 12B), and the degradation behavior of barrier layer 22B.

In contrast, the constructs of FIG. 4b do not include a barrier layer. Instead a network of micro-channels is formed by the matrix and/or encapsulation layers, thereby providing spatial separation between the encapsulation layer and the drug-loaded matrix layer. This network of micro-channels can be filled with a liquid which can serve as a carrier for the embedded substance(s) in the matrix layer. Such a liquid can also act as a carrier for other therapeutic agents (e.g., drugs in the liquid). Customization of the drug mixture directly before use can be performed by an end user.

Constructs as in FIG. 4c are similar to those of FIG. 4a, except that the barrier layer is laterally structured to form pockets of relatively rapidly degradable material (lightly shaded) separated by relatively slowly degradable material (unshaded). Such constructs can provide pulsed release of drugs (e.g., by altering the degradation lifetime of the rapidly degradable polymer from pocket to pocket). In this manner, a predetermined sequence of drug deliveries can be provided by a single polymer structure.

Constructs as in FIG. 4d include two matrix layers disposed on top of each other, with separated voids. The top matrix layer (e.g., layer 32B or layer 42A) is relatively rapidly degradable, and provides a burst release (layer 32B) or delayed burst release (layer 42A) of the drugs incorporated into its pattern. Substances from the bottom matrix layer 32E can be released in a pulsed release. Multiple matrix layers can be employed, each including the same or different substances, to provide controlled release of multiple therapeutic agents.

FIG. 4e shows embodiments having two matrix layers with physically connected voids. Release of both drugs is simultaneous. Release can be delayed by the encapsulation layer (e.g., layer 12E), or by the second matrix layer (e.g., layer 32B). As above, additional matrix layers can be added.

FIG. 4f shows embodiments where both top and bottom surfaces of a polymer multi-layer structure are utilized for drug release. The material being released can be the same on the two sides (e.g., matrix layer 44A) or can be different on the two sides (e.g., matrix layer 44B). Similarly, the barrier layers and encapsulation layers can be the same on both sides or can differ.

FIG. 4g shows an embodiment of the invention suitable for providing radiation therapy. In this case, an encapsulation layer 12A having no through holes is employed, to prevent the release of radioactive material while it is still active. A single structure can provide combined chemical and radiation therapy, where radioactive therapeutic agent(s) are enclosed in sealed voids (e.g., as in FIG. 4g), and chemical therapeutic agent(s) are enclosed in unsealed voids (e.g., as in FIGS. 4a-f).

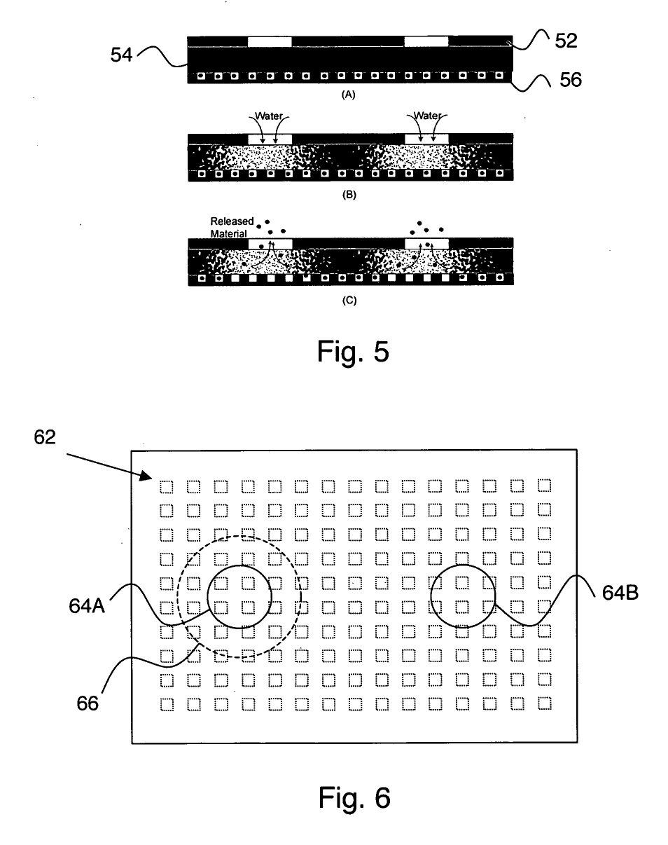

FIGS. 5a-c show an example of how an embodiment of the invention can operate in practice. In this example, an encapsulation layer 52 is disposed on top of a barrier layer 54, which is disposed on top of a matrix layer 56. In this example, all layers are made of biodegradable polymers. Typical feature dimensions are 100 μm diameter through holes in encapsulation layer 52 and 20 μm diameter voids in matrix layer 56. Encapsulation layer 52 and matrix layer 56 have in vivo lifetimes that are greater than therapy duration, so that their geometric features remain substantially unaffected by degradation during therapy. In contrast, barrier layer 54 has an in vivo lifetime that is shorter than therapy duration. Thus degradation of barrier layer 54 (FIG. 5b) permits release of the therapeutic agent(s) (FIG. 5c). Once therapy is complete, layers 52 and 56 degrade. As indicated above, drug release can be via diffusion, osmosis, or a combination of these mechanisms.

Diffusion limited release is driven by the concentration gradient across the partially or completely degraded barrier layer from high concentration (at the matrix layer) to low concentration (at the encapsulation layer). The top view of FIG. 6 is useful in considering the delivery rate in this case. Here matrix layer voids 62 are shown in dotted lines, while encapsulation layer holes 64A, 64B are shown in solid lines. As a simplified model of barrier layer degradation, it is assumed that barrier layer degradation proceeds by expansion of a circular boundary extending laterally in the barrier layer from each encapsulation layer through hole. Thus boundary 66 corresponds to hole 64A. Boundary 66 has a radius x, which increases as the barrier layer degrades (i.e., x is time-dependent).

The drug concentration gradient is approximately given by ρ/x, where ρ is the drug concentration at boundary 66. Here it is assumed that each drug reservoir is small compared to hole 64A (i.e., many voids 62 intersect with boundary 66), and that the drug concentration is negligible at the center of hole 64A. From Fick's law, the diffusion flux is then Dρ/x, where D is the diffusion constant. The release rate Q across boundary 66 (and out hole 64A) is given by Q=(Dρ/x)2π×h=2πhDρ, where h is the thickness of the barrier layer. For N identical holes, the total release rate Qₜₒₜ=2πNhDρ. This model shows zero-order (i.e., constant rate) release, since the x dependence of the flux is canceled by the x dependence of the boundary area. Let N=l²/d², where l² is the layer area, and d is the separation between holes (assumed disposed on a square lattice), which gives Qₜₒₜ=2πhDρl²/d². Thus increasing the hole separation d will decrease delivery rate. The barrier layer thickness h can also be used to control delivery rate, since increasing h increases the delivery rate.

Parameters of the encapsulation layer can also be used to select between diffusion limited release, osmosis driven release, or a combination thereof. It is helpful to define A as being the total area of all through holes in encapsulation layer 52. It is helpful to define parameters Aₘₐₓ and Aₘᵢₙ by

$$\begin{matrix}{A}_{min} = 5 \sqrt{l \frac{ⅆ V}{ⅆ t} \frac{η}{Δ {P}_{max}}} , \\ and & ( 1 ) \\ {A}_{max} = \frac{l}{F} {( \frac{ⅆ m}{ⅆ t} )}_{z} \frac{1}{DS} , & ( 2 )\end{matrix}$$

where l is the length of the opening (i.e., the thickness of encapsulation layer 52), dV/dt is the volume flux through the openings, η is the viscosity of the dispensed solution, ΔPₘₐₓ is the maximum allowed pressure difference between interior and exterior of the polymer structure, (dm/dt), is the zero-order osmosis driven delivery rate, S is the drug solubility, and F is a minimum ratio of osmotic delivery rate to diffusion delivery rate. If A<Aₘₐₓ, osmosis is the dominant delivery mechanism, and diffusion is negligible (it is recommended that the empirical factor F be ≧40 to ensure negligible diffusion). If A<Aₘᵢₙ, hydrostatic pressure can exceed the pressure limit ΔPₘₐₓ, so preferably Aₘᵢₙ<A<Aₘₐₓ.

The osmosis driven delivery rate is given by

$${( \frac{ⅆ m}{ⅆ t} )}_{z} = \frac{A}{h} k {π}_{s} S , \qquad ( 3 )$$

where πₛ is the osmotic pressure at saturation and k is the product of mechanical permeability and reflection coefficient. When the condition Aₘᵢₙ<A<Aₘₐₓ holds, the release rate is given by Eq. 3. Osmosis driven release can be performed with or without a barrier layer. If a barrier layer is not present, osmosis driven release commences as soon as the polymer structure is placed in a water-containing environment (e.g., after implantation). If a barrier layer is present, release can be diffusion limited as the barrier layer degrades, and can then become osmosis driven after complete degradation of the barrier layer.

Further embodiments and variations of the invention are described in the following examples.

### EXAMPLE 1

This example relates to release of a hydrophobic substance (specifically, the antibiotic tetracycline) at high rates. The polymer multi-layer structure is as shown in FIGS. 5a-c, where the barrier layer is a low molecular weight 50/50 poly (lactic-co-glycolic) acid (PLGA), and the encapsulation and matrix layers are 85/15 PLGA. The barrier layer thickness is 50 μm and the encapsulation layer thickness is 25 μm. The encapsulation layer through holes are 100 μm in diameter and are fabricated by hot embossing. The matrix layer voids are 20 μm squares having a depth of about 10 μm formed by hot embossing. Tetracycline is embedded into the matrix layer voids by screen printing. The layers are laminated by a thermal fusion process at a temperature higher than the glass transition temperatures of the layers and lower than the melting temperatures of the layers.

In this example, the layer parameters are designed to provide osmosis driven drug release. The barrier layer starts degrading after about one day in a water containing environment. The degradation mechanism for this polymer is bulk degradation, so that polymer fragments are formed during degradation. The increasing concentration of these fragments will lead to additional water uptake from the environment, and an increase in osmotic pressure.

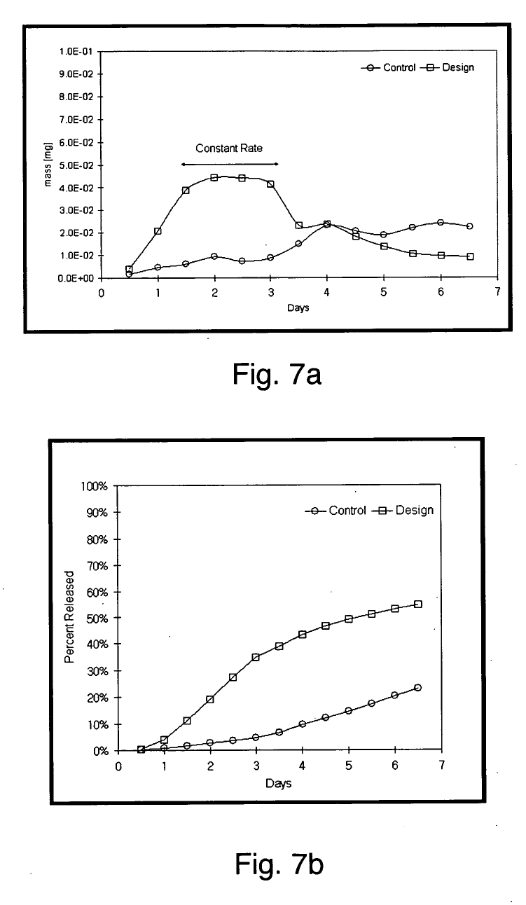

The release behavior for this structure in a phosphate buffer solution was experimentally studied. Samples were taken twice a day, and the concentration of released tetracycline was measured via fluorescence with a fluorescence plate reader. For this experiment, a control structure omitting the encapsulation layer was used for comparison. FIGS. 7a-b show tetracycline release as a function of time for these two cases. Here “control” labels the control device (i.e., no encapsulation layer) and “design” labels the sample device having an encapsulation layer. No initial drug release burst is apparent, due to the time required for barrier layer degradation. The sample device provides a high and approximately constant release rate for a significant time span (from about 1.5 days to about 3 days). After 3 days, about 50% of the total drug dose is delivered. Following this time period the rate decreases. This decreasing rate is consistent with the 1/(1+t)² behavior expected when the osmotic pressure starts dropping (due to a decrease in the concentration of polymer fragments from the degrading barrier layer). In comparison to the sample device, the control device show a low release rate, due to the low solvability of tetracycline in water. Furthermore, the delivery rate is not significantly constant, and instead appears to be affected by details of the degradation of the barrier layer.

### EXAMPLE 2

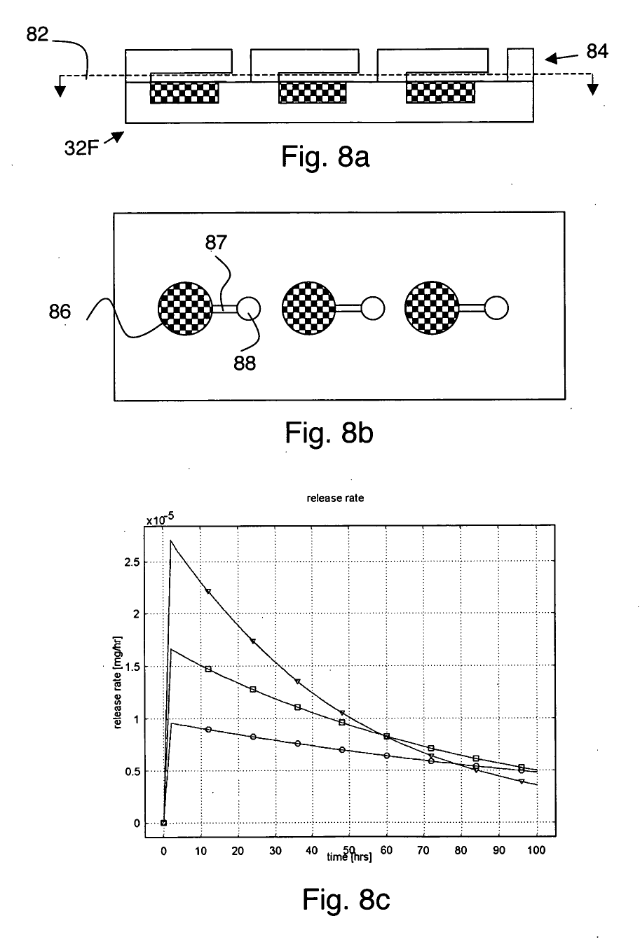

FIGS. 8a-b show an embodiment of the invention where drug-containing reservoirs are connected to an outer surface of a delivery device via channels. The system of reservoirs 86 and channels 87 is formed by patterns formed in a matrix layer 32F and an encapsulation layer 84. Upon bonding of these two layers, the reservoirs and channels are formed. Encapsulation layer 84 includes through holes 88. FIG. 8a shows a side view, while FIG. 8b shows a view along line 82 of FIG. 8a. The channels can be open or can be filled with a rapidly degradable polymer (i.e., having a lifetime less than therapy duration). Typical feature dimensions are as follows: reservoir diameter about 1 mm, reservoir height of about 100 μm, channel length about 1 cm, channel diameter between about 25 μm and about 50 μm, and encapsulation layer through hole diameter from about 200 μm to about 1 mm. As above, the delivery mechanism can be diffusion and/or osmosis. The delivery rate can be controlled by altering geometrical parameters of the patterns, especially the channel parameters. For example, delivery rate is decreased by increasing channel length and/or decreasing channel diameter.

FIG. 8c shows calculated drug delivery rates for embodiments according to FIGS. 8a-b having different channel lengths. On this plot, the triangles correspond to a channel length of 1 mm, the squares correspond to a channel length of 2 mm, and the circles correspond to a channel length of 3 mm. Increasing the channel length decreases the delivery rate.

One preferred embodiment of a reservoir-channel structure has an encapsulation layer of poly(ε-caprolactone-co-glycolide), a matrix layer of poly(ε-caprolactone-co-glycolide), and an agent including levobupivacaine, bupivacaine, lidocaine, and/or ropivacaine combined with or without anti-inflammatory agents. Another preferred embodiment of a reservoir-channel structure has an encapsulation layer of poly(lactide-co-glycolide), a matrix layer of poly(lactide-co-glycolide), and an agent including levobupivacaine, bupivacaine, lidocaine, and/or ropivacaine combined with or without anti-inflammatory agents.

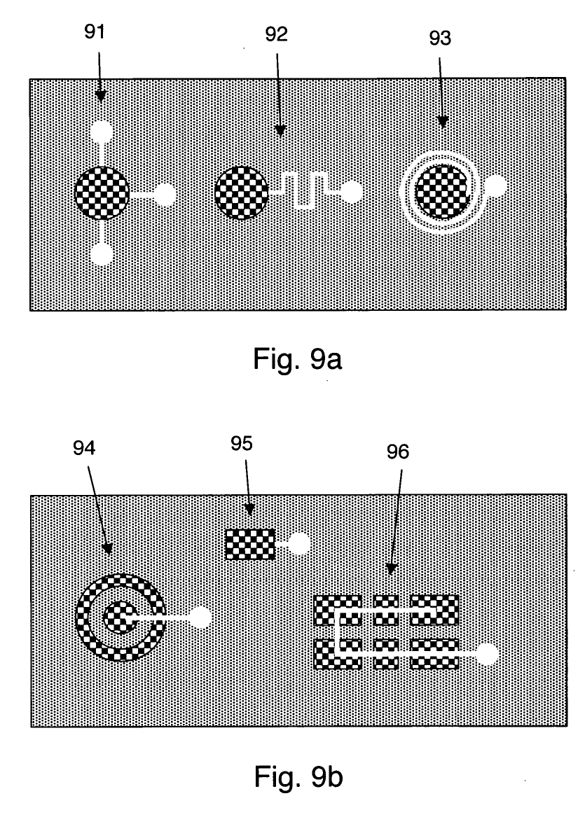

Many variations of channel-reservoir embodiments are possible. For example, FIG. 9a shows several channel variations, such as multiple channels leading to the same reservoir (91), a serpentine channel (92) and a spiral channel (93). FIG. 9b shows several reservoir configurations, such as a radially symmetric multi-compartment reservoir configuration (94), a rectangular reservoir (95) and another multi-compartment reservoir configuration (96). In general, the reservoirs and channels can have any shape, which provides a great deal of flexibility. In addition, since the reservoirs and channels can be made independent of one another, customizable delivery of multiple agents can be provided without having to account for interactions of agents within the delivery device. Other variations of channel-reservoir embodiments include having release openings on both sides of a device (analogous to the embodiments of FIG. 4f). For example, a drug reservoir can have a channel that connects to a hole that extends through the entire thickness of the polymer structure. In this manner, drug release from both sides of a polymer construct can be provided. In this example, the through holes can be formed after the layers of the polymer structure are bonded together (i.e., the reservoirs in the matrix layer are predetermined, while the through holes are not predetermined).

Channel reservoir embodiments can also be designed to provide osmotic and/or diffusive release, as considered in connection with Example 1, and more specifically in Eqs. 1 and 2. In this context, l and A in Eqs. 1 and 2 can be taken to be the channel length and channel cross sectional area respectively.

### EXAMPLE 3

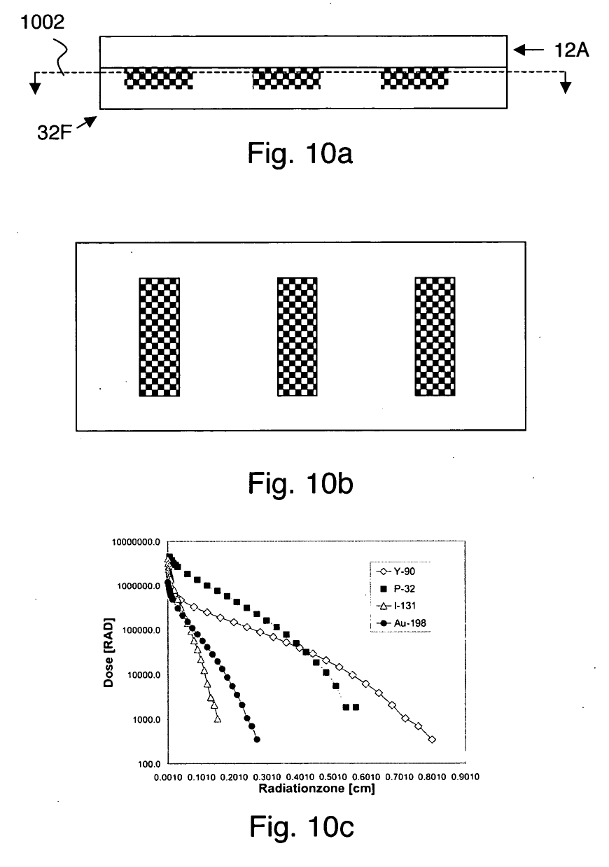

As indicated above, embodiments of the invention can be employed for radiation therapy. FIGS. 10a-b show an embodiment of the invention where the therapeutic agent is radioactive and encapsulation layer 12A is a solid layer having no through holes. Matrix layer 32F includes a radioactive therapeutic agent in its voids. FIG. 10b shows a view along line 1002 on FIG. 10a. These layers are preferably bio-degradable with an in vivo lifetime that is substantially longer than a duration of the therapy (i.e., greater than ten times the longest half life of any of the radioactive agents included in the matrix layer). In this manner, release of the agent is prevented while it is radioactive. Eventual in vivo release of spent radioactive agents is not problematic, if there is no significant chemical toxicity. Many radioactive agents decay to harmless substances (e.g., isotope P-32 becomes S). Preferably the therapeutic agent is a beta emitter having a half life of less than about 400 hours. Suitable therapeutic agents include Y-90 (half life 64.1 h), Au-198 (half life 64.704 h), P-32 (half life 342.96 h) and I-131 (half life 193.2 h).

The voids can have any shape. Preferably they are generally channel-shaped if the agents are to be loaded in liquid form, and are isolated voids if a solid agent is employed. Channel shaped voids preferably have a length between about 10 mm and about 60 mm, a width between about 20 μm and about 300 μm, and a height between about 25 μm and about 100 μm. It is important that the polymers employed for this application of the invention not be deleteriously affected by the radiation. Tests have been performed that indicate that PLGA is sufficiently unaffected by radiation.

FIG. 10c shows dose vs. distance for the embodiment of FIGS. 10a-b. Four isotopes are considered, and in each case, the assumed loading density is 1 mC/cm². An alternative way to compare these isotopes is to consider the loading density required to provide a typical therapeutic dose of 10 Gy (1000 rad), and the distance at which the 10 Gy dose is obtained, as in the following table.

| Isotope | mCi/cm² | Distance for 10 Gy | |
|---|---|---|---|
| Y-90 | 0.011325 | 0.28 | cm |
| P-32 | 0.004405 | 0.27 | cm |
| I-131 | 0.969456 | 0.15 | cm |
| Au-198 | 2.894595 | 0.27 | cm |

As indicated above, a key application of the invention is to structures which are implanted in the body, either separately or on an outer surface of some other implant (e.g., such as stents, catheters, and joint replacements). Such other implants can be temporary or permanent. In cases where a polymer structure of the invention is implanted by itself, or is affixed to another permanent implant, it is preferred for the matrix and encapsulation layers to degrade after completion of therapy. Alternatively, a polymer structure of the invention can be applied to a surface of an organism being treated (e.g., for transdermal drug delivery applications). In such cases, the matrix and encapsulation layers need not be biodegradable. Similarly, if a polymer structure of the invention is attached to a temporary implant, the matrix and encapsulation layers need not be biodegradable.

It will be appreciated that the device of the invention can be implanted using methods known in the art, including invasive, surgical, minimally invasive and non-surgical procedures. Depending on the subject, target sites, and agent(s) to be delivered, the microfabrication techniques disclosed herein can be adapted to make the delivery device of the invention of appropriate size and shape.

Although the preceding description relates to therapeutic applications, the invention is also applicable to non-therapeutic applications such as cell culturing and tissue engineering. Thus agents that can be controllably released by embodiments of the invention include therapeutic agents, cell culture agents and tissue engineering agents.

The invention is suitable for controlled delivery of any agent. By way of example, suitable agents include but are not limited to the following: nucleic acids; nucleotides; oligonucleotides; peptides; polypeptides; chemotherapeutic agents; thrombolytics; vasodilators; growth factor antagonists; free radical scavengers; biologic agents; radiopaque agents; radiolabelled agents; anti-coagulants; anti-angiogenesis drugs; angiogenesis drugs; PDGF-B and/or EGF inhibitors; riboflavin; tiazofurin; zafurin; ADP inhibitors; hosphodiesterase I11; lycoprotein II/IIIIa agents; adenosine reuptake inhibitors; healing and/or promoting agents; antiemetics; antinauseants; immunosuppressants; anti-inflammatories; anti-proliferatives; anti-migratory agents; anti-fibrotic agents; proapoptotics; calcium channel blockers; anti-neoplastics; antibodies; anti-thrombotic agents; anti-platelet agents; IIbIIIIa agents; antiviral agents; analgesia agents (e.g., bupivacaine, levobupivacaine, lidocaine, gabapentin, ketamin, clonidine, dextatomide, ropivacaine and derivations or combinations of any of these); antibiotic agents (e.g., tetracycline, adriamycine, penicillin, minocycline and derivations or combinations of any of these); anti-cancer agents and radio-sensitizers (e.g., branodeoxyuridine, myfermycine, cisplatin, gemcitabin, adiramycine, topotecan hydrochloride, paclitaxel, cisplatin, 5-fluorouracil, carmustine, interferon alpha, tamoxifen, tirapazamine, cytoxan and derivations or combinations of any of these); short half life radio-therapeutic agents (e.g., Y-90, P-32, I-131, Au-198); hormones and anti-hormonal agents (e.g., estrogens, steroids, androgens, progestins, dexa methasone, and thyroid and antithyroid drugs); growth factors (e.g., fibro blast growth factors, nerve growth factors, bone morphogenic protein, platelet derived growth factors, epidermal growth factors, vascular endothelial growth factors, transforming growth factors beta, and derivations or combinations of any of these); genes (e.g., DNA derivates); dermatological drugs; and ophthalmologic drugs.

The terms apparatus and device are used interchangeably throughout to refer to implantable and non-implantable structures of this invention.

Therapeutic Applications

The apparatus of the invention can be utilized to deliver drugs, proteins, peptides, nucleic acids, including nucleic acid vectors, nucleotides, autologous or heterologous cells, or any therapeutic capable agents. The apparatus and methods of the invention can be utilized in vivo, ex vivo, or in vitro, such as in cell culture.

The devices described herein are suitable for the treatment of diseases. It would be appreciated that the disease being treated is related to the drug contained in the device. Diseases, conditions or disorders that can be treated with the devices described herein include autoimmune diseases, inflammatory diseases, cardiovascular diseases, conditions with pain symptoms, neuronal diseases, metabolic diseases, cancer anemia, infectious agents such as bacteria, virus or parasites, psychological disorders or mental disease (e.g., attention deficit disorder, anxiety, depression) or, nutritional disorders (e.g., obesity, malnutrition or anemia), hematological disorders or diseases (e.g., hypertension, coagulation), bone diseases, and ulcers.

The devices can be used to administer agents therapeutically to achieve a therapeutic benefit or prophylactically to achieve a prophylactic benefit. By therapeutic benefit is meant eradication or amelioration of the underlying disorder being treated. For prophylactic benefit, the agents may be administered to a patient at risk of developing a disease or to a patient reporting one or more of the physiological symptoms of such a disease, even though a diagnosis may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication.

The devices described herein that are suitable for use in the methods of the present invention include devices wherein the drug is contained in a therapeutically or prophylactically effective amount, i.e., in an amount effective to achieve therapeutic or prophylactic benefit, as previously discussed. Of course, the actual amount effective for a particular application will depend, inter alia, on the condition being treated and the route of administration. Determination of an effective amount is well within the capabilities of those skilled in the art.

In one aspect of the invention, the therapeutic capable agents may be selected from a group consisting of immunosuppressants, anti-inflammatories, anti-proliferatives, anti-migratory agents, anti-fibrotic agents, proapoptotics, calcium channel blockers, anti-neoplastics, antibodies, anti-thrombotic agents, anti-platelet agents, IIbIIIIa agents, antiviral agents, and a combination thereof. Specific examples of therapeutic capable agent include: mycophenolic acid, mycophenolate mofetil, mizoribine, methylprednisolone, dexamethasone, Certican™, rapamycin, Triptolide™, Methotrexate™, Benidipine™, Ascomycin™, Wortmannin™, LY294002, Camptothecin™, Topotecan™, hydroxyurea, Tacrolimus™ (FK 506), cyclophosphamide, cyclosporine, daclizumab, azathioprine, prednisone, Gemcitabine™, derivatives, pharmaceutical salts and combinations thereof.

Additional therapeutic capable agents may comprise at least one compound selected from the group consisting of anti-cancer agents; chemotherapeutic agents; thrombolytics; vasodilators; antimicrobials or antibiotics; antimitotics; growth factor antagonists; free radical scavengers; biologic agents; radio therapeutic agents; radiopaque agents; radiolabelled agents; anti-coagulants such as heparin and its derivatives; anti-angiogenesis drugs such as Thalidomide™; angiogenesis drugs; PDGF-B and/or EGF inhibitors; anti-inflammatories including psoriasis drugs; riboflavin; tiazofurin; zafurin; anti-platelet agents including cyclooxygenase inhibitors such as acetylsalicylic acid, ADP inhibitors such as clopidogrel (e.g., Plavix™) and ticlopdipine (e.g., ticlid™), hosphodiesterase I11 inhibitors such as cilostazol (e.g., Pletal™)g, lycoprotein II/IIIIa agents such as abciximab (e.g., Rheopro™); eptifibatide (e.g., Integrilin™), and adenosine reuptake inhibitors such as dipyridmoles; healing and/or promoting agents including anti-oxidants, nitrogen oxide donors; antiemetics; antinauseants; tripdiolide, diterpenes, triterpenes, diterpene epoxides, diterpenoid epoxide, triepoxides, or tripterygium wifordii hook F(TWHF), SDZ-RAD, RAD, RAD666, or 40-O-(2-hydroxy)ethyl-rapamycin, derivatives, pharmaceutical salts and combinations thereof.

Anticancer Agents

In some aspects of the invention, the apparatus of the invention are utilized to deliver an anti-tumor capable therapeutic agent. An anti-tumor therapeutic capable agent is a molecule which decreases or prevents a further increase in growth of a tumor and includes anti-cancer agents such as Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride, and Taxol.

Another example of anti-cancer agents includes Topoisomerase I inhibitors. This class is structurally related to the natural compound camptothecin, which is derived from the Chinese Camptotheca acuminata plant. Topoisomerase I inhibitors differ from topoisomerase II inhibitors, such as etoposide, in that they bind to the topoisomerase-DNA complex; cell death ensues when the DNA helix cannot rebuild after uncoiling. The two most promising compounds in this class are irinotecan and topotecan; such anticancer agents can be used in treating a variety of cancers, including colorectal cancer, small-cell lung cancer, ovarian cancer, stomach cancer, cervical cancer, skin cancer, liver cancer, kidney cancer, pancreatic cancer, testicular cancer, prostate cancer, nasophangeal cancers, or buccal cancers.

Polypeptides

In another aspect of the invention, the therapeutic capable agent is a bioactive protein or peptide. Examples of such bioactive protein or peptides include a cell modulating peptide, a chemotactic peptide, an anticoagulant peptide, an antithrombotic peptide, an anti-tumor peptide, an anti-infectious peptide, a growth potentiating peptide, and an anti-inflammatory peptide. Examples of proteins include antibodies, enzymes, steroids, growth hormone and growth hormone-releasing hormone, gonadotropin-releasing hormone, and its agonist and antagonist analogues, somatostatin and its analogues, gonadotropins such as luteinizing hormone and follicle-stimulating hormone, peptide T, thyrocalcitonin, parathyroid hormone, glucagon, vasopressin, oxytocin, angiotensin I and II, bradykinin, kallidin, adrenocorticotropic hormone, thyroid stimulating hormone, insulin, glucagon and the numerous analogues and congeners of the foregoing molecules. The therapeutic agents may be selected from insulin, antigens selected from the group consisting of MMR (mumps, measles and rubella) vaccine, typhoid vaccine, hepatitis A vaccine, hepatitis B vaccine, herpes simplex virus, bacterial toxoids, cholera toxin B-subunit, influenza vaccine virus, bordetela pertussis virus, vaccinia virus, adenovirus, canary pox, polio vaccine virus, plasmodium falciparum, bacillus calmette geurin (BCG), klebsiella pneumoniae, HIV envelop glycoproteins and cytokins and other agents selected from the group consisting of bovine somatropine (sometimes referred to as BST), estrogens, androgens, insulin growth factors (sometimes referred to as IGF), interleukin I, interleukin II and cytokins. Three such cytokins are interferon-α, interferon-β and tuftsin.

In one embodiment a cell modulating peptide is selected from the group consisting of an anti-integrin antibody fragment, a cadherin binding peptide, a bone morphogenic protein fragment, and an integrin binding peptide. Preferably the cell modulating peptide is a integrin binding peptide which is selected from the group consisting of RGDC, RGEC, RGDT, DGEA, DGEAGC, EPRGDNYR, RGDS, EILDV, REDV, YIGSR, SIKVAV, RGD, RGDV, HRNRKGV, KKGHV, XPQPNPSPASPVVVGGGASLPEFXY, and ASPVVVGGGASLPEFX. The peptides also may be any functionally active fragment of the proteins disclosed herein as being bioactive molecules useful according to the invention. In another embodiment the chemotactic peptide is selected from the group consisting of functionally active fragments of collagen, fibronectin, laminin, and proteoglycan. In yet another embodiment the anti-tumor peptide is selected from the group consisting of functionally active fragments of protein anti-tumor agents. The anti-infectious peptide is selected from the group consisting of functionally active fragments of the protein anti-infectious agents according to another embodiment. In another embodiment the growth potentiating peptide is selected from the group consisting of functionally active fragments of PDGF, EGF, FGF, TGF, NGF, CNTF, GDNF, and type I collagen related peptides. According to another embodiment the anti-inflammatory peptide is selected from the group consisting of functionally active fragments of anti-inflammatory agents.

Other bioactive peptides useful according to the invention may be identified through the use of synthetic peptide combinatorial libraries such as those disclosed in Houghton et al., Biotechniques, 13(3):412-421 (1992) and Houghton et al., Nature, 354:84-86 (1991) or using phage display procedures such as those described in Hart, et al., J. Biol. Chem. 269:12468 (1994). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively to a specific molecule such as a cell surface receptor are obtained by selecting those phages which express on their surface a ligand that binds to the specific molecule. Ligands that possess a desired biological activity can be screened in known biological activity assays and selected on that basis. These phages then are subjected to several cycles of reselection to identify the peptide-expressing phages that have the most useful characteristics. Typically, phages that exhibit the binding characteristics (e.g., highest binding affinity or cell stimulatory activity) are further characterized by nucleic acid analysis to identify the particular amino acid sequences of the peptides expressed on the phage surface and the optimum length of the expressed peptide to achieve optimum biological activity. Alternatively, such peptides can be selected from combinatorial libraries of peptides containing one or more amino acids. Such libraries can further be synthesized which contain non-peptide synthetic moieties which are less subject to enzymatic degradation compared to their naturally-occurring counterparts. U.S. Pat. No. 5,591,646 discloses methods and apparatuses for biomolecular libraries which are useful for screening and identifying bioactive peptides. Methods for screening peptides libraries are also disclosed in U.S. Pat. No. 5,565,325.

Peptides obtained from combinatorial libraries or other sources can be screened for functional activity by methods known in the art. For instance when the peptide is a cell modulating peptide, and in particular an integrin binding peptide, one of ordinary skill in the art can easily determine whether the peptide will modulate bone cell activity by performing the in vitro studies set forth in example 2 to measure osteoblast differentiation. Likewise, similar experiments can be conducted for other types of cells using cell specific markers of differentiation or growth. The type of assay of course, used for a particular peptide depends on the source of the peptide. For instance if a peptide is a fragment of an anti-tumor molecule, the peptide should be tested for functional activity in an anti-tumor assay. Those of skill in the art can easily choose an appropriate assay for testing functionality of a particular peptide.

The bioactive molecules useful according to the invention are commercially available from many sources and methods for making these molecules also are well known in the art. Bioactive peptides and proteins may easily be synthesized or produced by recombinant means. Such methods are well known to those of ordinary skill in the art. Peptides and proteins can be synthesized for example, using automated peptide synthesizers which are commercially available. Alternatively the peptides and proteins can be produced by recombinant techniques by incorporating the DNA expressing the peptide into an expression vector and transforming cells with the expression vector to produce the peptide. In such an example, the DNA expressing vector is the therapeutic capable agent that is delivered utilizing the apparatus of the invention. Alternatively, the DNA expression vector, can itself be present in a eukaryotic cell that is housed in the implantable device of the invention. Such cells can be autologous so as to obviate any immunotoxicity. Alternatively, heterologous cells may be used where such cells are engineered to reduce, minimize or eliminate immunotoxicity in the recipient animal.

Of course it will be apparent to one of skill in the art that the device of the invention, when engineered secretory cells are disposed in the reservoir layer, in order to preclude immunotoxicity, conventional immunosuppressive agents may be used during the course of treatment. Examples of such immunosuppressive agents, include but are not limited to such as cyclophosphamide, cyclosporin, tacrolimus (FK506), azathioprine, prednisone, methylprednisolone, prostaglandin, and steroids, can also be administered, as is known in the art, in conjunction with the implant to quash the tissue rejection response and promote immunotolerance. In one aspect of the invention the implantable device of the invention will provide the additional immunosuppressive in addition to the cells producing the transgene product that is therapeutic.

Alternatively, the device will function as a sieve which allows therapeutic proteins produced from cells contained in the reservoir portions to exit, but precluding the cells' exposure to an animal's immune system. In such an example. Designs for such implantable devices comprising cells producing therapeutic agents are known, in the art, for example as disclosed in U.S. Pat. No. 6,743,626, the disclosure of which is incorporated by reference herein.

Additional bioactive molecules can be therapeutic capable agents used in the device and methods of the invention. For example, IL-1, of which there may be several forms, such as IL-1-alpha and IL-1-beta, can be delivered to target cells or tissue in a subject or in vitro in cell culture assays. Preferred cytokines for use in the method and compositions of the invention are lymphokines, i.e., those cytokines which are primarily associated with induction of cell differentiation and maturation of myeloid and possibly other hematopoietic cells. A preferred lymphokine is IL-1. Other such lymphokines include, but are not limited to, G-CSF, M-CSF, GM-CSF, Multi-CSF (IL-3), and IL-2 (T-cell growth factor, TCGF). IL-1 appears to have its effect mostly on myeloid cells, IL-2 affects mostly T-cells, IL-3 affects multiple precursor lymphocytes, G-CSF affects mostly granulocytes and myeloid cells, M-CSF affects mostly macrophage cells, GM-CSF affects both granulocytes and macrophage. Other growth factors affect immature platelet (thrombocyte) cells, erythroid cells, and the like.

In other aspects of the invention, cytokines can be used alone or in combination to protect against, mitigate and/or reverse myeloid or hematopoietic toxicity associated with cytotoxic agents. Examples of possible combinations include IL-1+GC-CSF, IL-1+IL-3, G-CSF+IL-3, IL-1+ platelet growth factor and the like. Certain combinations will be preferred, depending on the maturation state of the target cells or tissues to be affected, and the time in the course of cytotoxic action that the protective agent needs to be administered. For example, in patients with depression of several hematopoietic cell types (e.g., myeloid, lymphoid and platelet), a combination of IL-1+IL-3/and/or platelet growth factor is preferred, while more severe depression of the myeloid series may require such combinations as IL-1+G-CSF. Certain cytotoxic agents have greater compromising effects on particular hematopoietic elements, either because of the nature of the agent or the dosage necessary to achieve a therapeutic effect, and the appropriate choice, dosage and mode of administration of cytokine(s) will follow from such effects. The device of the invention can be custom designed to deliver a particular cytokine or growth factor based on the desired treatment and underlying condition.

In other aspects of the invention, the implantable device is designed to deliver proteins such as antibodies. Antibodies themselves can be used as cytotoxic agents, either by virtue of their direct, e.g., complement mediated, action upon, e.g., invading microorganisms or proliferating tumor cells, or by an indirect mode, e.g., through mobilization of T-cells (e.g., killer cells), an action known as antibody-directed cellular cytotoxicity (ADCC). Such antibody cytotoxicity, denoted herein as unconjugated cytotoxic antibody therapy, can also result in compromise of elements of the hematopoietic system, and such adverse side effects can be prevented, mitigated and/or reversed with adjunctive cytokine therapy. In other words, the implantable device can concomitantly release cytokine therapeutic agents to provide a alleviate any of the preceding adverse side affects.

In yet other aspects, the device will deliver protein factors that promote angiogenesis. Angiogenesis, the growth of new blood vessels in tissue, has been the subject of increased study in recent years. Such blood vessel growth to provide new supplies of oxygenated blood to a region of tissue has the potential to remedy a variety of tissue and muscular ailments, particularly ischemia. Primarily, study has focused on perfecting angiogenic factors such as human growth factors produced from genetic engineering techniques. It has been reported that injection of such a growth factor into myocardial tissue initiates angiogenesis at that site, which is exhibited by a new dense capillary network within the tissue. Schumacher et al., “Induction of Neo-Angiogenesis in Ischemic Myocardium by Human Growth Factors”, Circulation, 1998; 97:645-650. Angiogenic factors include but are not limited to: VEGF, Hypoxia inducible factor (HIF), fibroblast growth factor (FGF), HO-1, SOD, NOSII, NOSIII, placental growth factor (PLGF), TGF.beta., angiopoietin-1, bFGF, and macrophage chemoattractant protein-1 (MCP-1), as well as functional derivatives or combinations thereof.

Nucleic Acids

Nucleic acids include nucleotides; oligonucleotides; and their art-recognized and biologically functional analogs and derivatives including, for example, oligonucleotide analogs having phosphorothioate linkages. Additional examples, include antisense RNA, siRNA, microRNA, DNA/RNA hybrids, and nucleic acid containing vectors. Examples of vectors include andenoviral vectors, adenoviral associated vectors, retroviral vectors, and/or plasmid vectors. The device of the invention can utilize recombinant DNA technology known in the art. Further, recombinant genes useful in the methods of the present invention include known nucleic acid molecules which encode a protein of interest, such protein being useful in the treatment of the subject.

In addition nucleic acids include nucleic acid molecules that encode proteins, nucleic acids that include a gene or multiple genes (e.g., including introns and exons), that encode fusion proteins, that encode selectable markers or can comprise vectors that containing any one or combination of the preceding.

In some aspects of the invention the nucleic acid vectors are deposited in the apparatus of the invention and are delivered to a target cell or tissue. In other aspects, such vectors can encode a therapeutic protein or antisense mRNA. In yet other aspects of the invention, one or more vectors each encoding a different therapeutic capable agent delivered to cells or tissue via the device of the invention.

Therefore, the device of the invention will controllably release vectors to effectuate gene delivery, such as in gene therapy. Gene delivery may be either endogenously or exogenously controlled. Examples of endogenous control include promoters which are sensitive to a physiological signal such as hypoxia or glucose elevation. Exogenous control systems involve gene expression controlled by administering a small molecule drug. Examples include tetracycline, doxycycline, ecdysone and its analogs, RU486, chemical dimerizers such as rapamycin and its analogs, etc.

In an alternative aspect of the invention, the device can deliver the small molecule drug, such as those in the preceding paragraph, where the device is utilized to deliver the vector and the inducible agent (e.g., small molecule drug), the vector alone or some combination thereof.

Vectors include derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by P J Southern and P Berg, J Mol Appl Genet 1:327-341 (1982); Subramini et al., Mol Cell. Biol. 1:854-864 (1981), Kaufinann and Sharp, J Mol. Biol. 159:601-621 (1982); Scahill et al., PNAS USA 80:4654-4659 (1983) and Urlaub and Chasin PNAS USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMulV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6(7):608-614 (1988), which is hereby incorporated by reference.

Recombinant retroviral vectors into which multiple genes may be introduced may also be used according to the methods of the present invention. As described by Eglitis and Andersen, supra, vectors with internal promoters containing a cDNA under the regulation of an independent promoter, e.g. SAX vector derived from N2 vector with a selectable marker (noe.sup.R) into which the cDNA for human adenosine deaminase (hADA) has been inserted with its own regulatory sequences, the early promoter from SV40 virus (SV40) may be designed and used in accordance with the methods of the present invention by methods known in the art.

In some aspects of the invention, the vectors comprising recombinant nucleic acid molecules are first introduced (e.g., transfected) into cells, which cells are deposited in the apparatus of the invention. For example, the vectors comprising the recombinant nucleic acid molecule are incorporated, i.e. infected, into the BM-MNCs by plating ˜5e5 BM-MNCs over vector-producing cells for 18-24 hours, as described by Eglitis and Andersen BioTechniques 6(7):608-614 (1988), which is hereby incorporated by reference, and subsequently said cells are deposited into the reservoir portion of the device.

In some aspects of the invention the nucleic acid molecule encodes proteins such as growth factors, including but not limited to, VEGF-A, VEGF-C PlGF, KDR, EGF, HGF, FGF, angiopoietin-1, and cytokines. In additional preferred embodiments, the nucleic acid molecule encodes endothelial nitric oxide synthases eNOS and iNOS, G-CSF, GM-CSF, VEGF, aFGF, SCF (c-kit ligand), bFGF, TNF, heme oxygenase, AKT (serine-threonine kinase), HIF.alpha. (hypoxia inducible factor), Del-1 (developmental embryonic locus-1), NOS (nitric oxide synthase), BMP's (bone morphogenic proteins), SERCA2a (sarcoplasmic reticulum calcium ATPase), .beta.sub.2-adrenergic receptor, SDF-1, MCP-1 other chemokines, interleukins and combinations thereof. In additional aspects of the invention, the apparatus/device of the invention comprises genes which may be delivered in the autologous BM-MNCs using the methods of the present invention include but are not limited to nucleic acid molecules encoding factor VIII/von Willebrand, factor IX and insulin, NO creating genes such as eNOS and iNOS, plaque fighting genes thrombus deterrent genes, for example. Therefore, in such an example, the apparatus of the invention contains cells that secrete the therapeutic agent into the reservoir layer of the apparatus, wherefrom the therapeutic agent exits from the apparatus into the surrounding cells (e.g., in vitro or in vivo). It will be appreciated that the preceding growth factors can also be delivered in the form of synthesized or recombinant proteins.

In mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the AQP1 gene product in infected hosts. (See e.g., Logan & Shenk, Proc. Natl. Acad. Sci. USA 8 1:3655-3659 (1984)). Specific initiation signals can also be required for efficient translation of inserted therapeutic nucleotide sequences. These signals include the ATG initiation codon and adjacent sequences. In cases where an entire therapeutic gene or cDNA, including its own initiation codon and adjacent sequences, is inserted into the appropriate expression vector, no additional translational control signals can be needed. However, in cases where only a portion of the therapeutic coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See e.g., Bittner et al., Methods in Enzymol, 153:516-544 (1987)).

Tissue Engineering

In some aspects of the invention, the outer layer of the invention comprises a substrate surface defining a tissue contacting surface, whereby the surface is disposed with polypeptides or peptides which are cell/tissue growth potentiating. Examples of such polypeptides/peptides include peptide PDGF, EGF, FGF, TGF, NGF, CNTF, GDNF, VEGF and type I collagen peptides, or functionally active fragments and/or combinations thereof.

In one aspect of the invention, a peptide-coated implantable device of the invention is for enhancing and/or accelerating tissue growth. For example, the device can be used to promote bone growth in areas of damaged bone or in bone replacement surgery. Bone and joint replacement surgeries are commonly used, for instance, to relieve pain, improve function, and enhance the quality of life for patients with medical conditions caused by osteoarthritis, rheumatoid arthritis, post-traumatic degeneration, avascular necrosis, and other aging-related conditions.

The device of the invention which is coated with bioactive peptides that enhance or accelerate bone growth will significantly improve the ability of an implant to remain attached to the bone surface. Preferred integrin binding peptides which perform this function are RGDC, RGEC, RGDT, DGEA, DGEAGC, EPRGDNYR, RGDS, EILDV, REDV, YIGSR, SIKVAV, RGD, RGDV, and HRNRKGV. Concomitantly, the device of the invention can release or deliver a therapeutic capable agent that enhances or promotes osteocyte proliferation and differentiation, for whatever period of time deemed necessary to effectuate therapy.

In yet other aspects the device of the device of the invention provides for a fibrin matrix comprising short peptides covalently crosslinked thereto, as well as bioactive factors. Such factors can be attached to the outer surface of the device 52 (FIG. 5). The fibrin matrix may be further defined as a fibrin gel. The matrix chosen is fibrin, since it provides a suitable three dimensional structure for tissue growth and is the native matrix for tissue healing. The crosslinking would be accomplished enzymatically by using the native Factor XIII to attach the exogenous factors to the gels. In order to do this, a sequence that mimics a crosslinking site can be incorporated into the peptide so that the enzyme recognized and crosslinked it into the matrix.

Novel activity will be conferred to these fibrin gels by adding a peptide sequence, or other bioactive factor, which is delivered via the device of the invention. These materials may be useful in the promotion of healing and tissue regeneration, in the creation of neurovascular beds for cell transplantation and in numerous other aspects of tissue engineering. Hence, the invention in yet other aspects provides compositions created and adapted for these specific uses.

Cell Culture

In some aspects of the invention, the device or methods of the invention can be utilized in cell culture or tissue culture assays. For example, the device is utilized in a cell culture to release a particular agent in a controlled manner to monitor the effects of such an agent on cells or tissue cultures. For example, the apparatus of the invention can be utilized in a method of screening different agents to determine the mechanisms, by which such compounds induce cell differentiation, e.g., such as in studying effects on stem cells. Methods of utilizing cell and tissue culture are known in the art, such as U.S. Pat. No. 7,008,634 (using cell growth substrates with tethered cell growth effector molecules); U.S. Pat. No. 6,972,195 (culturing potentially regenerative cells and functional tissue organs in vitro); U.S. Pat. No. 6,982,168 or 6,962,980 (using cell culture to assay compounds for treating cancer); U.S. Pat. No. 6,902,881 (culturing techniques to identify substances that mediate cell differentiation); U.S. Pat. No. 6,855,504 (culturing techniques for toxicology screening); or U.S. Pat. No. 6,846,625 (identifying validated target drug development using cell culture techniques), the disclosure of each of which is herein incorporated by reference. The device of the invention is readily adaptable to such cell culturing techniques as would be evident to one of ordinary skill in the art.

Analgesia Agents

In some aspects of the invention, the apparatus of the invention is utilized to deliver a therapeutic capable agent that is an analgesic. Such agents include but are not limited to Bupivacaine and derivations such as Hydrochloride, Bupivacain, Levobupivacain, Lidocaine and derivations, Gabapentin and derivations, Ketamin and derivations, Clonidine and derivations, Dextatomide and derivations, Ropivacaine and derivations, or combinations thereof.

Antibiotics

In some aspects of the invention, the apparatus of the invention are utilized to deliver an antibiotic, or an anti-infectious therapeutic capable agent. Such anti-infectious agents reduce the activity of or kills a microorganism and includes Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride, as well as derivations, and combinations thereof.

Anti-Inflammatory

In some aspects of the invention, the apparatus of the invention are utilized to deliver an anti-inflammatory therapeutic capable agent. Such an anti-inflammatory agent reduces an inflammatory response and includes steroidal and non-steroidal compounds; Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; s Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Additional nonsteroidal anti-inflammatory agents that may be used include, but are not limited to, aspirin, diclofenac, flurbiprofen, ibuprofen, ketorolac, naproxen, and suprofen. In a further variation, the antiinflammatory agent is a steroidal anti-inflammatory agent.

Anticoagulant

In some aspects of the invention, the apparatus of the invention are utilized to deliver a therapeutic capable agent that is an anticoagulant. Such an anticoagulant agent is a molecule that prevents clotting of blood and includes but is not limited to Ancrod; Anticoagulant Citrate Dextrose Solution; Anticoagulant Citrate Phosphate Dextrose Adenine Solution; Anticoagulant Citrate Phosphate Dextrose Solution; Anticoagulant Heparin Solution; Anticoagulant Sodium Citrate Solution; Ardeparin Sodium; Bivalirudin; Bromindione; Dalteparin Sodium; Desirudin; Dicumarol; Heparin Calcium; Heparin Sodium; Lyapolate Sodium; Nafamostat Mesylate; Phenprocoumon; Tinzaparin Sodium; Warfarin Sodium.

Antithrombotic

In some aspects of the invention, the apparatus of the invention are utilized to deliver a therapeutic capable agent that is antithrombotic. An antithrombotic molecule as used herein is a molecule that prevents formation of a thrombus and includes but is not limited to Anagrelide Hydrochloride; Bivalirudin; Dalteparin Sodium; Danaparoid Sodium; Dazoxiben Hydrochloride; Efegatran Sulfate; Enoxaparin Sodium; Ifetroban; Ifetroban Sodium; Tinzaparin Sodium; Trifenagrel.

Radio Therapeutic

In some aspects of the invention, radioisotopes can be delivered via the implantable device of the invention. For example, it is well known in the art that various methods of radionuclide therapy can be used for the treatment of cancer and other pathological conditions, as described, e.g., in Harbert, “Nuclear Medicine Therapy”, New York, Thieme Medical Publishers, 1987, pp. 1-340. A clinician experienced in these procedures will readily be able to adapt the implantable device described herein to such procedures to mitigate or treat disease amenable to radioisotope therapy thereof.

In some aspects the radio isotopes include but are not limited to isotopes and salts of isotopes with short half life: such as Y-90, P-32, I-131, Au 198. Therefore in one aspect of the invention, the implantable device can be utilized to deliver radioisotopes.

It is also well known that radioisotopes, drugs, and toxins can be conjugated to antibodies or antibody fragments which specifically bind to markers which are produced by or associated with cancer cells, and that such antibody conjugates can be used to target the radioisotopes, drugs or toxins to tumor sites to enhance their therapeutic efficacy and minimize side effects. Examples of these agents and methods are reviewed in Wawrzynczak and Thorpe (in Introduction to the Cellular and Molecular Biology of Cancer, L. M. Franks and N. M. Teich, eds, Chapter 18, pp. 378-410, Oxford University Press, Oxford, 1986), in Immunoconjugates. Antibody Conjugates in Radioimaging and Therapy of Cancer (C.-W. Vogel, ed., 3-300, Oxford University Press, New York, 1987), in Dillman, R. O. (CRC Critical Reviews in Oncology/Hematology 1:357, CRC Press, Inc., 1984), in Pastan et al. (Cell 47:641, 1986), in Vitetta et al. (Science 238:1098-1104, 1987) and in Brady et al. (Int. J. Rad. Oncol. Biol. Phys. 13:1535-1544, 1987). Other examples of the use of immunoconjugates for cancer and other forms of therapy have been disclosed, inter alia, in Goldenberg, U.S. Pat. Nos. 4,331,647, 4,348,376, 4,361,544, 4,468,457, 4,444,744, 4,460,459, 4,460,561 and 4,624,846, and in Rowland, U.S. Pat. No. 4,046,722, Rodwell et al., U.S. Pat. No. 4,671,958, and Shih et al., U.S. Pat. No. 4,699,784, the disclosures of all of which are incorporated herein in their entireties by reference.

In an alternative aspect of the invention the implantable device can be utilized in therapy to deliver antibodies conjugated with radioisotopes. Much of radioisotope therapy is effected with beta emitters, alpha emitters and/or with the radioisotope generated in situ by neutron activation of Boron-10 atoms (resulting in alpha emission from the unstable nuclide produced by neutron absorption.) P-32-orthophosphate can be administered via the device of the invention. For example, the device can be designed to effect controlled release of doses of about 3 to 10 mCi, doses between 0.1 to 1.5 mCi, or doses of 7 to 10 mCi as clinically required, and during a time course for therapy.

In alternative aspects of the invention, these doses can be increased by from about 10% to about 35%, preferably 15 to 25%, by simultaneous administration of continuous or intermittent (i.e., controlled release) doses of about 5 to 20 ug of IL-1, more preferably 5-10 ug IL-1, extending to several days post-radionuclide therapy. Similarly, Re-186-under simultaneous and post-therapy administration of IL-1 (5-10 ug) alone or in combination with IL-3 (2-10 ug), repeated several times during a 1-2 week therapy course.

Further, in other alternative aspects of the invention, one or more implantable device can be implanted, each of which can controllably release a different therapeutic capable agent (e.g., radioisotopes). Of course as noted herein through out, each device can release a combination of different therapeutic capable agents (e.g., radioisotopes and cytokines).

Dermatological

In some aspects of the invention, the device can be utilized transdermally to deliver therapeutic capable agents in treatment of dermatological disorders. For example, a low molecular weight compound (e.g., a pain relieving substance or mixture of pain relieving substances) is transdermally delivered to cells of the body using an embodiment of a transdermal delivery system of the invention. Examples of such therapeutic agents include but are not limited to: non-steroidal anti-inflammatory drugs (NSAIDs) that are frequently administered systemically such as ibuprofen (2-(isobutylphenyl)-propionic acid); methotrexate (N-[4-(2,4 diamino 6-pteridinyl-methyl]methylamino]benzoyl)-L-glutamic acid); aspirin (acetylsalicylic acid); salicylic acid; diphenhydramine (2-diphenylmethoxy)-NN-dimethylethylamine hydrochloride); naproxen (2-naphthaleneacetic acid, 6-methoxy-9-methyl-, sodium salt, (−)); phenylbutazone (4-butyl-1,2-diphenyl-3,5-pyrazolidinedione); sulindac-(2)-5-fuoro-2-methyl-1-[[p-(methylsulfinyl)phenyl]methylene-]-1H-indene-3-acetic acid; diflunisal (2′,4′-difluoro-4-hydroxy-3-biphenylcarboxylic acid); piroxicam (4-hydroxy-2-methyl-N-2-pyridinyl-2H-1,2-benzothiazine-2-carboxamide 1,1-dioxide, an oxicam; indomethacin (1-(4-chlorobenzoyl)-5-methoxy-2-methyl-H-indole-3-acetic acid); meclofenamate sodium (N-(2,6-dichloro-m-tolyl)anthranilic acid, sodium salt, monohydrate); ketoprofen (2-(3-benzoylphenyl)-propionic acid; tolmetin sodium (sodium 1-methyl-5-(4-methylbenzoyl-1H-pyrrole-2-acetate dihydrate); diclofenac sodium (2-[(2,6-dichlorophenyl)amino]benzeneatic acid, monosodium salt); hydroxychloroquine sulphate (2-{[4[(7-chloro-4-quinolyl)amino]pentyl]ethylamino}ethanol sulfate (1:1); penicillamine (3-mercapto-D-valine); flurbiprofen ([1,1-biphenyl]-4-acetic acid, 2-fluoro-alphamethyl-, (+−.)); cetodolac (1-8-diethyl-13,4,9, tetra hydropyrano-[3-4-13]indole-1-acetic acid; mefenamic acid (N-(2,3-xylyl)anthranilic acid; and diphenhydramine hydrochloride (2-diphenyl methoxy-N,N-di-methyletthmine hydrochloride).

In yet further aspects of the invention, steroid hormone preparations, retinoid preparations, immunosuppressive agents, and antibiotics can be used for the treatment of eczema, atopic dermatitis, psoriasis, pruritus, ichthyosis, acne, inflammation, erythema, and bacterial infections accompanying with dysfunctions of the skin barrier.

In additional aspects anti-inflammatory therapeutic agents can be utilized with the device of the invention. Generally, anti-inflammatory agents inhibit protein kinase C (referred to hereinafter as PKC), and many PKC activity-inhibiting agents have been developed and employed as anti-inflammatory agents. In the biochemical pathway of inflammation induction, PKC activity increases due to exogenous stimuli, followed by an increase in phospholipase D (referred to hereinafter as PLD) activity, thereby proceeding to inflammation.

In other aspects, the therapeutic agent for treatment of skin diseases is provided, having a sphingolipid long-chain base and lysophosphatidic acid. In some embodiments, the sphingolipid long-chain base can be present at a percentage (by weight) from about 0.01 to 5.0%. In some embodiments, the lysophosphatidic acid can be present at from about 0.001 to 1.0%. The sphingolipid long-chain base can be, for example, phytosphingosine, acetylphytosphingosine, tetraacetyl phytosphingosine, hexanoylphytosphingosine, or acetylphytosphingosine phosphate.

In accordance with another aspect of the present invention, the above and other objects can be accomplished by the provision of a therapeutic composition for a broad spectrum of skin diseases, comprising 30 to 90% by weight of a conventional substrate or a carrier for topical application; 0.01 to 5% by weight of sphingolipid long-chain base; 0.001 to 1% by weight of lysophosphatidic acid; and 1 to 40% by weight of organic or inorganic additives.

Preferably, the sphingolipid long-chain base is one or more selected from the group consisting of phytosphingosine, acetylphytosphingosine, tetraacetyl phytosphingosine, hexanoylphytosphingosine and acetylphytosphingosine phosphate. It is preferable that the organic additives may contain ceramide, cholesterol and fatty acid at a weight ratio of 40 to 60%:20 to 30%:20 to 30%, pursuant to the composition of normal skin. In some embodiments, ceramide used herein may include ceramide 3, ceramide 6, and a mixture thereof, and its stereochemical composition is the same as in skin lipids.

In some embodiments, the lysophosphatidic acid used herein may be selected from the group consisting of lyso-stearoyl phosphatidic acid (18:0), lyso-oleoyl phosphatidic acid (18:1), lyso-palmitoyl phosphatidic acid (16:0) and natural lyso-phosphatidic acid derived from egg yolk or beans. In accordance with another aspect of the present invention, there is provided a therapeutic composition for a broad spectrum of skin diseases, including atopic dermatitis, eczema, psoriasis with hyperkeratosis, skin inflammation, pruritus, bacterial infection, acne, and wounds.

As an active ingredient of the composition according to the invention, sphingolipid long-chain base can be used instead of steroid hormone preparations or retinoid preparations having an anti-inflammatory effect, immunosuppressive agents having an effect of alleviating skin irritation, and antibiotics. Controlled delivery using the device of the invention can provide chronic therapy thus preventing harshly scratched wounds due to severe pruritus, and fissures in the skin should be healed.

It is important to note that a device of the invention can also be designed of a scale to be utilized for topical delivery, such as in combination with an adhesive band or patch. In addition, “topical” as used herein includes applications where a device of the invention is implanted under the dermis, in the gastro intestinal tract, or in the vasculature of a subject.

Ophthalmologic

In another aspect of the invention, the device can be implanted in an ocular region. Delivery to the eye of a therapeutic amount of an active agent can be difficult, if not impossible, especially for drugs with short plasma half-lives since the exposure of the drug to intraocular tissues is limited. A more efficient way of delivering a drug to treat an ocular condition is to place the drug directly in the eye. In one broad aspect of the invention, the drug delivery device is sized and adapted for placement into an eye, for example into one of an anterior chamber of an eye and a posterior chamber of an eye.

In other words, the device of the invention can be microfabricated to an appropriate scale for implantation into any cell/tissue target area in a given animal, preferably a human. Techniques for implanting devices into the eye are known in the art. Weber et al., U.S. patent application Ser. No. 10/246,884, Pub. No. U.S.200410054374 A1, describes methods for delivering ocular implants into an eye of a patient; Wong, U.S. Pat. No. 5,824,072 discloses implants for introduction into a suprachoroidal space or an avascular region of the eye, and describes a methylcellulose (i.e., non-biodegradable) implant comprising dexamethasone. Weber et al. and Wong are incorporated by reference herein.

Therapeutic, active agents that may be used in the systems and methods of the present invention, such as for treatment of ocular disease/disorders, include, but are not limited to (either by itself or in combination with another active agent): ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; .beta.-adrenergic blocker or .beta.-adrenergic agonist, e.g. ephidrine, epinephrine, etc.; aldosereductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, antiglaucoma agents, such as brimonidine (Alphagan and Alphagan P), acetozolamide, bimatoprost (Lumigan), timolol, timolol maleate, mebefunolol; memantine; alpha-2 adrenergic receptor agonists; 2ME2; anti-neoplastics, such as vinblastine, vincristine, interferons; alpha., beta. and .gamma., antimetabolites, such as folic acid analogs, purine analogs, and pyrimidine analogs; immunosuppressants such as azathiprine, cyclosporine and mizoribine; miotic agents, such as carbachol, mydriatic agents such as atropine, etc., protease inhibitors such as aprotinin, camostat, gabexate, vasodilators such as bradykinin, etc., and various growth factors, such epidermal growth factor, basic fibroblast growth factor, nerve growth factors, and the like.

In one aspect of the invention, cortisone, dexamethasone, fluocinolone, hydrocortisone, methylprednisolone, prednisolone, prednisone, and triamcinolone, and their derivatives, are preferred steroidal anti-inflammatory agents. In another aspect of the invention, the steroidal anti-inflammatory agent is dexamethasone. In another aspect of the invention, the biodegradable implant includes a combination of two or more steroidal anti-inflammatory agents.

Other agents may be employed in the formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

Ocular disease that can be treated utilizing the implantable device of the invention include An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. An anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye.

In further aspects of the invention treatable ocular diseased include aposterior conditiona, where an aposterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because the therapeutic goal is to prevent the loss of or reduce the occurrence of loss of vision due to damage to or loss of retinal cells or optic nerve (i.e. neuroprotection).

Implantation

It will be appreciated that the device of the invention can be implanted using methods known in the art, including invasive, surgical, minimally invasive and non-surgical procedures. Depending on the subject, target sites, and agent(s) to be delivered the microfabrication techniques disclosed herein, can be adapted to make the delivery device of the invention of appropriate size and shape. The devices described herein are suitable for use in various locations in the body. For example, they can be implanted on the surface of the skin, under the skin, or in or near internal tissues or organs. The devices in some embodiments are located in or near a gastro-intestinal tract, airway tissue or organ, cardiovascular tissue or organ, or neuronal tissue or organ. Other examples of target sites for implantation include but are not limited to the eye, pancreas, kidney, liver, stomach, muscle, heart, lungs, lymphatic system, thyroid gland, pituitary gland, ovaries, prostate, skin, endocrine glands, ear, breast, urinary tract, brain or any other site in an animal.

For example, regarding implantation in the eye, suitable sites for implantation in the eye include the anterior chamber, posterior chamber, vitreous cavity, suprachoroidal space, subconjunctiva, episcleral, intracorneal, epicorneal and sclera. Suitable sites extrinsic to the vitreous comprise the suprachoroidal space, the pars plana and the like. The suprachoroid is a potential space lying between the inner scleral wall and the apposing choroid. Elements in accordance with the present invention that are introduced into the suprachoroid may deliver drugs to the choroid and to the anatomically apposed retina, depending upon the diffusion of the drug from the implant, the concentration of drug comprised in the implant and the like.

Additional methods and procedures for implanting a device of the invention are known in the art, such as disclosed in U.S. Pat. Nos. 7,013,177; 7,008,667; 7,006,870; 6,965,798; 6,963,771; 6,585,763; 6,572,605; or 6,419,709, the disclosure of each of which is herein incorporated by reference.

1. Apparatus comprising:
a matrix layer comprising a matrix polymer having a microstructured matrix layer spatial pattern having voids, wherein all geometrical details of the matrix layer spatial pattern are substantially predetermined;
one or more agents disposed in the voids; and
an encapsulation layer comprising an encapsulation polymer and disposed to cover the matrix layer spatial pattern.

2. The apparatus of claim 1, wherein said matrix polymer comprises a polymer selected from the group consisting of: aliphatic polyesters, copoly(ether-esters), polyalkylene oxalates, polyamides, polyorthoesters, polyoxaesters, poly(anhydrides), poly(dimethylsiloxane), silicone elastomers, polyurethane, poly(tetrafluoroethylene), polyethylene, polysulfone, poly(methyl methacrylate), poly(2-hydroxyethyl methacrylate), polyacrylonitrile, polyamides, polypropylene, poly(vinyl chloride), poly(ethylene-co-(vinyl acetate)), polystyrene, poly(vinyl pyrrolidine), saccharides, cellulose, chitin, dextran, proteins, collagen, albumin, acrylates, acrylamides, poly(acryl acid), polyacrylamide, poly(1-hydroxyethyl methacrylate), poly(ethylene glycol), yellow wax, petrolatum cholesterol, stearyl alcohol, white wax, white petrolatum, methylparaben, propylparaben, sodium lauryl sulfate, and mixtures, dispersions or co-polymers thereof.

3. The apparatus of claim 1, wherein said encapsulation polymer comprises a polymer selected from the group consisting of: aliphatic polyesters, copoly(ether-esters), polyalkylene oxalates, polyamides, polyorthoesters, polyoxaesters, poly(anhydrides), poly(dimethylsiloxane), silicone elastomers, polyurethane, poly(tetrafluoroethylene), polyethylene, polysulfone, poly(methyl methacrylate), poly(2-hydroxyethyl methacrylate), polyacrylonitrile, polyamides, polypropylene, poly(vinyl chloride), poly(ethylene-co-(vinyl acetate)), polystyrene, poly(vinyl pyrrolidine), and mixtures or co-polymers thereof.

4. The apparatus of claim 1, wherein said matrix layer and said encapsulation layer are both biodegradable, whereby said apparatus has an in vivo lifetime.

5. The apparatus of claim 4, wherein said in vivo lifetime is greater than a duration of said therapy, whereby delivery of said therapy is substantially independent of degradation of said matrix layer and said encapsulation layer.

6. The apparatus of claim 1, further comprising one or more additional matrix layers, each additional matrix layer having a predetermined spatial pattern including voids, wherein voids in each matrix layer include different therapeutic agents, whereby controlled delivery of multiple therapies is provided.

7. The apparatus of claim 1, wherein said one or more therapeutic agents comprises one or more chemical agents.

8. The apparatus of claim 7, wherein said matrix layer and said encapsulation layer have thicknesses between about 50 μm and about 150 μm.

9. The apparatus of claim 7, further comprising a barrier layer disposed between and in contact with said encapsulation layer and said matrix layer.

10. The apparatus of claim 7, wherein said barrier layer is biodegradable or solvable in tissue fluid and has a barrier layer lifetime less than a duration of said therapy.

11. The apparatus of claim 7, wherein said barrier layer comprises a material selected from the group consisting of: aliphatic polyesters, copoly(ether-esters), polyalkylene oxalates, polyamides, polyorthoesters, polyoxaesters, poly(anhydrides), saccharides, cellulose, chitin, dextran, proteins, collagen, albumin, acrylates, acrylamides, poly(acryl acid), polyacrylamide, poly(1-hydroxyethyl methacrylate), and poly(ethylene glycol), and mixtures, dispersions or co-polymers thereof.

12. The apparatus of claim 7, wherein said encapsulation layer has an encapsulation layer spatial pattern comprising through holes and wherein all geometrical details of the encapsulation layer spatial pattern are substantially predetermined.

13. The apparatus of claim 12, wherein a delivery rate of said one or more chemical agents as a function of time is predetermined, in part, by said encapsulation layer spatial pattern.

14. The apparatus of claim 13, wherein said delivery rate is primarily diffusion-limited.

15. The apparatus of claim 13, wherein said delivery rate is primarily osmosis-driven.

16. The apparatus of claim 12, wherein said matrix layer spatial pattern and said encapsulation layer spatial pattern combine to form reservoirs containing said therapeutic agent and channels for regulating delivery of said therapy, wherein the channels extend from the reservoirs to said through holes.

17. The apparatus of claim 16, wherein said channels include a biodegradable or solvable material having a lifetime less than a duration of said therapy.

18. The apparatus of claim 16, wherein at least one of said reservoirs includes two or more compartments.

19. The apparatus of claim 16, wherein said reservoirs have a diameter of about 1 mm and a height of about 100 μm.

20. The apparatus of claim 16, wherein said channels have a length less than about 3 cm and have a diameter between about 25 μm and about 50 μm.

21. The apparatus of claim 16, wherein said through holes have a diameter from about 200 μm to about 1 mm.

22. The apparatus of claim 1, wherein said encapsulation layer includes no through holes, and wherein said one or more therapeutic agents comprises one or more radioactive agents, each having a half life, whereby said therapy comprises radiotherapy.

23. The apparatus of claim 22, wherein said matrix layer and said encapsulation layer are both biodegradable, and wherein said matrix layer and said encapsulation layer each have an in vivo lifetime greater than about 10 times the longest of said half lives.

24. The apparatus of claim 22, wherein said voids are generally channel-shaped and have a length between about 10 mm and about 60 mm, a width between about 20 μm and about 300 μm, and a height between about 25 μm and about 100 μm.

25. The apparatus of claim 22, wherein said one or more therapeutic agents comprise a beta emitter having a half life of less than about 400 hours.

26. The apparatus of claim 1, wherein said one or more agents are selected from the group consisting of therapeutic agents, cell culture agents, tissue engineering agents or combinations thereof.

27. A method for providing therapy, the method comprising:
providing a polymer structure including:
a matrix layer comprising a matrix polymer having a microstructured matrix layer spatial pattern having voids; and
an encapsulation layer comprising an encapsulation polymer and disposed to cover the matrix layer spatial pattern;
wherein all geometrical details of the matrix layer spatial pattern are substantially predetermined;
providing one or more therapeutic agents disposed in the voids; and
delivering the polymer structure to an organism being treated.

28. The method of claim 27, wherein said delivering the polymer structure comprises applying the polymer structure to a surface of said organism.

29. The method of claim 27, wherein said delivering the polymer structure comprises implanting the polymer structure in said organism.

30. The method of claim 29, wherein said polymer structure is disposed on an outer surface of an implant.

31. The method of claim 30, wherein said implant is selected from the group consisting of stents, catheters, and joint replacements.

32. The method of claim 27, wherein said providing one or more therapeutic agents disposed in the voids is performed shortly prior to said delivering the polymer structure.

33. The method of claim 27, wherein said one or more therapeutic agents are provided in liquid form, and wherein said providing one or more therapeutic agents disposed in the voids comprises loading said voids via capillary action.

34. The method of claim 27, wherein said therapy is selected from the group consisting of: delivery of antibiotics for periodontitis; delivery of medication for glaucoma treatment; delivery of agents for skin treatment; transdermal delivery of drugs or medications; delivery of growth factors, peptides, or DNA for wound healing, skin tissue repair, peripheral or central nervous system repair, skeletal or muscle tissue repair, vascular tissue regeneration, and/or controlled differentiation of stem cells; delivery of pain relief agents and/or antibiotics for post-operative treatment; temporary or permanent implantation; and local delivery of anti-cancer medication, radio-sensitizer and/or radiation for cancer treatment.

35. A method for providing controlled release of an agent, the method comprising:
providing a polymer structure including:
a matrix layer comprising a matrix polymer having a microstructured matrix layer spatial pattern having voids; and
an encapsulation layer comprising an encapsulation polymer and disposed to cover the matrix layer spatial pattern;
wherein all geometrical details of the matrix layer spatial pattern are substantially predetermined; and
providing one or more agents disposed in the voids.
